(19) United States
(12) Patent Application Publication
(10) Pub. No.: US 2023/0390093 A1
(43) Pub. Date: Dec. 7, 2023

(54) HAND MOVEMENT CONTROL APPARATUS

(71) Applicant: Brooke Wheeler, Whitby (CA)

(72) Inventor: Brooke Wheeler, Whitby (CA)

(21) Appl. No.: 17/805,588

(22) Filed: Jun. 6, 2022

(51) Int. Cl. A61F 5/01

(52) U.S. Cl. CPC A61F 5/0118; A61F 2005/0197

(57) ABSTRACT

A hand movement control apparatus that is operably coupled to the wrist and hand of a user wherein the hand movement control apparatus inhibits a range of motion to eliminate hyperextension of the fingers from an involuntary muscular movement. The present invention includes a wrist member that is circumferentially disposed around the wrist of the user. A plurality of movement restriction members extend from the wrist member to fingertip members that are mounted to the end of the fingers of the user. The movement restriction members are manufactured from an elastic material and inhibit a range of motion without application of a muscular force that must be voluntary. The movement restriction members are additionally anchored to the inner surface of the fingers utilizing ring members wherein the ring members are surroundably mounted to each finger of the user. The hand movement control apparatus is adjustable in size.

### FIELD OF THE INVENTION

The present invention relates generally to medical assistance devices, more specifically but not by way of limitation, an apparatus that is configured to be secured to a hand of a user wherein the present invention is operable to assist a user execute movement of their fingers and wrists within a proper range of motion so as to avoid potential injury to those afflicted with connective tissue disorders such as but not limited to Ehlers-Danlos syndrome.

### BACKGROUND

As is known in the art, Ehlers-Danlos syndrome is a connective tissue disorder that effects the patients skin, joints and blood vessel walls. Connective tissue is a complex mixture of proteins and other substances that provide strength and elasticity to the underlying structures in the human body. Individuals who have Ehlers-Danlos syndrome usually have overly flexible joints and stretchy, fragile skin. This can become a problem for the patient when they have a wound that requires stitches because the skin often isn't strong enough to hold them. Another symptom of Ehlers-Danlos is overly flexible joints and a difficulty in controlling the movement of certain joints such as but not limited to the joints in the hand and fingers. Because the connective tissue that holds joints together is looser in a patient afflicted with Ehlers-Danlos the joints in their hand and other places can move far past the normal range of motion. Joint pain and dislocations are common and the ability for individuals to control this range of motion is severely diminished. Overly flexible joints can result in joint dislocations and early-onset arthritis.

There is no known cure for Ehlers-Danlos and the treatments can include physical therapy with medicinal therapies only being supportive in nature. The excess mobility is difficult to control from a muscular perspective by the afflicted patient. Without the aid of a device, many patients are unable to control movements such as but not limited to hand and finger movements. These patients have a difficult time performing normal activities such as but not limited to writing and operation of hand-held items.

Accordingly, there is a need for a device that can be operably coupled to a hand wherein the present invention is configured to provide range of motion control for the fingers of the wearer so as to inhibit hyperextension thereof.

### SUMMARY OF THE INVENTION

It is the object of the present invention to provide a joint movement control device that is configured to be releasably secured to a hand of a user in order to provide range of motion control of the fingers wherein the present invention includes a wrist member circumferentially disposed around the wrist of the wearer.

Another object of the present invention is to provide a hand movement control apparatus operable to limit range of motion of the joints of the hand of a wearer wherein the present invention further includes a plurality of movement restriction members operably coupled to the wrist member.

A further object of the present invention is to provide a joint movement control device that is configured to be releasably secured to a hand of a user in order to provide range of motion control of the fingers wherein the movement restriction members include a first end and a second end.

Still another object of the present invention is to provide a hand movement control apparatus operable to limit range of motion of the joints of the hand of a wearer wherein the movement restriction members are manufactured from an elastic material.

An additional object of the present invention is to provide a joint movement control device that is configured to be releasably secured to a hand of a user in order to provide range of motion control of the fingers wherein the present invention further includes fingertip members.

Yet a further object of the present invention is to provide a hand movement control apparatus operable to limit range of motion of the joints of the hand of a wearer wherein the fingertip members are configured to be placed over the top of each finger of the hand and inserted thereinto.

Another object of the present invention is to provide a joint movement control device that is configured to be releasably secured to a hand of a user in order to provide range of motion control of the fingers wherein the second end of the movement restriction members are secured to the fingertip members.

Still another object of the present invention is to provide a hand movement control apparatus operable to limit range of motion of the joints of the hand of a wearer wherein the present invention further includes a plurality of ring members.

An additional object of the present invention is to provide a joint movement control device that is configured to be releasably secured to a hand of a user in order to provide range of motion control of the fingers wherein the plurality of ring members are surroundably secured to the fingers of the hand of the wearer.

Still a further object of the present invention is to provide a hand movement control apparatus operable to limit range of motion of the joints of the hand of a wearer wherein the movement restriction members are secured to the ring members such that the movement restriction members are intermediate the ring members and the fingers of the wearer.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a side perspective view of the present invention secured to an exemplary hand; and

FIG. 2 is a side perspective view of the present invention secured to an exemplary hand showing a range limited movement; and

FIG. 3 is a front view of the present invention secured to an exemplary hand.

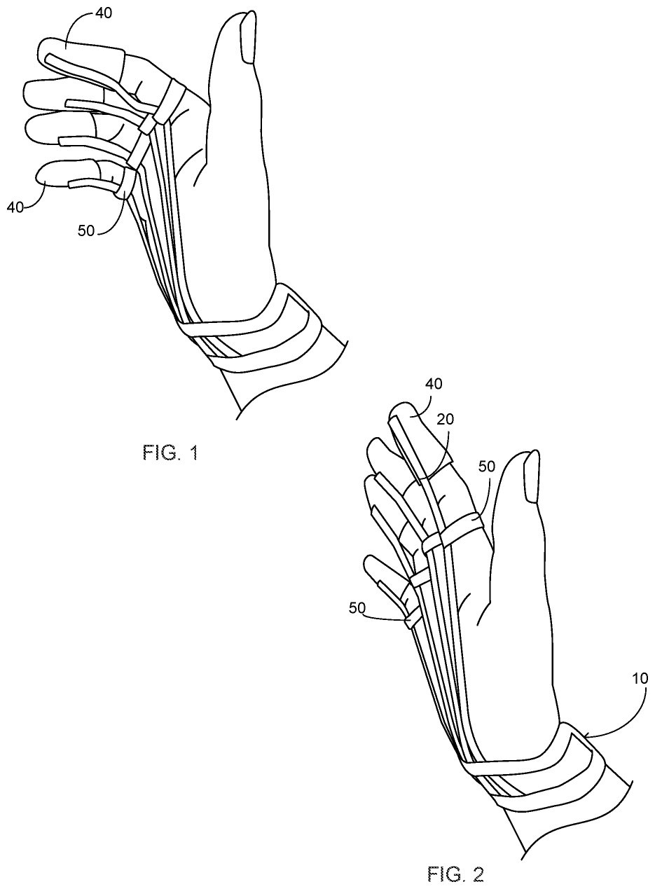

### DETAILED DESCRIPTION

Referring now to the drawings submitted herewith, wherein various elements depicted therein are not necessarily drawn to scale and wherein through the views and figures like elements are referenced with identical reference numerals, there is illustrated a hand movement control apparatus 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted as a part hereof, the hand movement control apparatus 100 includes a wrist member 10. The wrist member 10 is manufactured from cloth or other suitable material and is configured to be operably coupled to the wrist of a user of the hand movement control apparatus 100. The wrist member 10 circumferentially surrounds the wrist of the user and is adjustable in diameter through conventional techniques so as to accommodate alternate wrist sizes. The wrist member 10 provides an anchor for the first end of the movement restriction members 20 as is further discussed herein. The wrist member 10 includes an upper portion 12 and a lower portion 14 having a void 16 therebetween in the preferred embodiment illustrated therein. The upper portion 12 and lower portion 14 are identical in size and are both configured to be adjustable in diameter in order to be secured to alternate wrist sizes. The configuration of the upper portion 12 and lower portion 14 provides two anchoring points for the first end of the movement restriction members 20. While the preferred embodiment of the wrist member 10 includes an upper portion 12 and a lower portion 14, it is contemplated within the scope of the present invention that the wrist member 10 could be a single band having sufficient width to provide anchoring of the first ends of the movement restriction members 20.

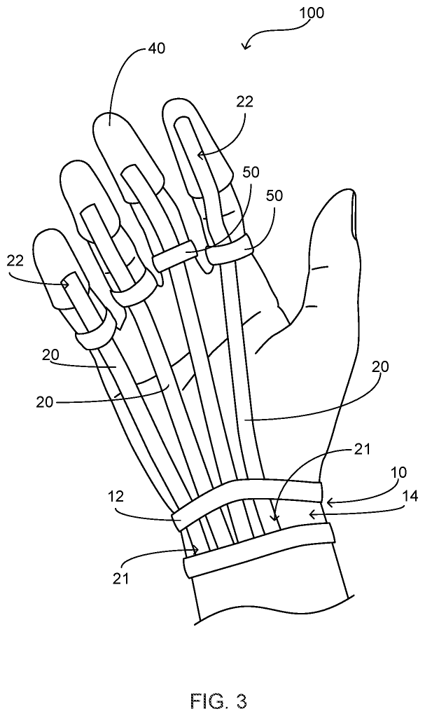

The hand movement control apparatus 100 includes a plurality of movement restriction members 20 that are operable to inhibit hyperextension and offer movement control of the phalanges of the hand of the user of the hand movement control apparatus 100. The movement restriction members 20 are manufactured from an elastic material and are elongated in shape having a first end 21 and second end 22. The movement restriction members 20 are operably coupled to the wrist member 10 and the fingertip members 40 providing tension therebetween. As the movement restriction members 20 are elastic during an exemplary movement to open the hand as illustrated herein in FIG. 2, the movement restriction members 20 will provide resistance to this movement and inhibit without application of a force that provides sufficient stretching of the movement restriction members 20. This inhibits undesired involuntary movement of the fingers that can result in painful hyperextension thereof. An application of force to open the hand while wearing the hand movement control apparatus 100 requires a voluntary muscular movement. While no technique is particularly illustrated herein, it is contemplated within the scope of the present invention that the movement restriction members 20 are adjustable in length in order to facilitate the ability for the hand movement control apparatus 100 to be fitted onto hands of alternate sizes.

The hand movement control apparatus 100 further includes fingertip members 40. The fingertip members 40 are manufactured from a suitable durable material such as but not limited to cloth. The fingertip members 40 are disposed on the ends of the fingers of the user of the hand movement control apparatus 100. In a preferred embodiment, the fingertip members 40 are tube shaped having a cavity that is of suitable size to receive therein a fingertip. The fingertip members 40 are manufactured of a sufficient length so as to extend down the fingers providing a sufficient operable connection therewith to provide anchoring of the second end 22 of the movement restriction members 20. It should be understood within the scope of the present invention that the fingertip members 40 could be provided in alternate sizes. Furthermore, it is contemplated within the scope of the present invention that the fingertip members 40 could be constructed in alternate manners in order to be secured to the fingertips of the user and provide anchoring of the second ends 22 of the movement restriction members 20.

A plurality of ring members 50 are circumferentially mounted to the fingers of the hand of the wearer of the hand movement control apparatus 100. The ring members 50 are manufactured from a suitable material such as but not limited to cloth. The ring members 50 are secured to the fingers of the hand of the wearer proximate the midpoint of the finger. The ring members 50 secure the movement restriction members 20 such that the movement restriction members 20 are intermediate the ring members 50 and the fingers of the user of the hand movement control apparatus 100. This positioning of the ring members 50 and operable coupling with the movement restriction members 20 provides the desired positioning of the movement restriction members 20 such that the movement restriction members 20 stay adjacent to the palm and inner finger surface of the wearer. It is contemplated within the scope of the present invention that the ring members 50 are adjustable in size utilizing suitable techniques in order to be secured to fingers of alternate sizes.

While the embodiment of the hand movement control apparatus 100 illustrated herein shows movement restriction members 20 along the palm and inside surface of the fingers, it is contemplated within the scope of the present invention that the hand movement control apparatus 100 could be provided in an embodiment that includes movement restriction members 20 along the back of the palm and back surface of the fingers in order to control a contraction movement of the hand. Additionally, it should be understood within the scope of the present invention that while the hand movement control apparatus 100 is illustrated and discussed herein as being operably coupled to all four fingers on a hand that the hand movement control apparatus 100 could be configured with the elements discussed herein on less than four fingers to include but not limited to a single finger.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A hand movement control apparatus operable to inhibit range of motion from involuntary muscular movement wherein the hand movement control apparatus comprises:
a wrist member, said wrist member being circumferentially disposed around a wrist of a user;
at least one fingertip member, said at least one fingertip member being secured to a fingertip of a finger of the user;
a movement restriction member, said movement restriction member being elongated in shape, said movement restriction member having a first end and a second end, said first end of said movement restriction member being secured to said wrist member, said second end of said movement restriction member being secured to said at least one fingertip member; and
wherein said movement restriction member is of a length that is operable to inhibit a range of motion of the finger.

2. The hand movement control apparatus as recited in claim 1, wherein the at least one fingertip member is tube shaped having a cavity configured to have a fingertip inserted thereinto.

3. The hand movement control apparatus as recited in claim 2, and further including a ring member, said ring member being annular in shape, said ring member being circumferentially disposed around the finger having the at least one fingertip member.

4. The hand movement control apparatus as recited in claim 3, wherein the movement restriction member is manufactured from an elastic material.

5. The hand movement control apparatus as recited in claim 4, wherein a portion of the movement restriction member is intermediate an inner surface of the ring member and the finger to which the ring member is secured.

6. A hand movement control apparatus operably coupled to a hand of a user so as to limit a range of motion of fingers on the hand wherein the hand movement control apparatus comprises:
a wrist member, said wrist member being circumferentially disposed around a wrist of the user;
four fingertip members, said four fingertip members being tubular in shaped having a first end and a second end, said first end having an opening providing access to a cavity, said four fingertip members configured to have fingertips of the user inserted into the cavity so as to secure thereto;
four movement restriction members, said four movement restriction members having a first end and a second end, said movement restriction members being elongated in shape, said four movement restriction members having a first end and a second end, said first end of said four movement restriction members being secured to said wrist member, said second end of said four movement restriction members being secured to said four fingertip members; and
wherein the movement restriction members are configured to limit a range of motion of the fingers.

7. The hand movement control apparatus operably coupled to the hand of the user so as to limit a range of motion of the fingers on the hand as recited in claim 6, wherein said four movement restriction members are manufactured from an elastic material.

8. The hand movement control apparatus operably coupled to the hand of the user so as to limit a range of motion of the fingers on the hand as recited in claim 7, and further including four ring members, said four ring members being secured to the fingers of the user proximate a midpoint thereof.

9. The hand movement control apparatus operably coupled to the hand of the user so as to limit a range of motion of the fingers on the hand as recited in claim 8, wherein the four ring members secure a portion of each of the four movement restriction members wherein the portion is intermediate the four ring members and the fingers of the user.

10. The hand movement control apparatus operably coupled to the hand of the user so as to limit a range of motion of the fingers on the hand as recited in claim 9, wherein the wrist member and four fingertip members are manufactured from a cloth material.
